(19) United States
(12) Patent Application Publication
(10) Pub. No.: US 2010/0286672 A1
(43) Pub. Date: Nov. 11, 2010

(54) Laser Scalpel

(75) Inventors: Rudolf Walker, Neunkirchen am Brand (DE); Gerd Volland, Erlangen-Bruck (DE); Reinhardt Thyzel, Heroldsberg (DE)

Correspondence Address: Workman Nydegger;1000 Eagle Gate Tower, 60 East South Temple, Salt Lake City, UT, 84111, US

(73) Assignee: A.R.C. LASER GMBH, Nurnberg (DE)

(21) Appl. No.: 12/718,689

(22) Filed: Mar. 5, 2010

(30) Foreign Application Priority Data
Mar. 6, 2009 (DE) 102009011587.0

(51) Int. Cl. A61B 18/20

(52) U.S. Cl. 606/3

(57) ABSTRACT

The invention is based on the object of specifying a laser scalpel, by means of which a high proportion of the laser light coupled into the scalpel body can be provided on the scalpel edge or is able to emerge therefrom. According to a first aspect, the invention is based on the idea of coupling the laser light into the scalpel body at an acute angle, with respect to the surface normal of the coupling-in surface, that is to say with respect to the direction or axis perpendicular or normal to the coupling-in surface.

### BACKGROUND OF THE INVENTION

1. Technical Field

The invention relates to a laser scalpel.

2. Background and Relevant Art

WO 01/87176 A1 has disclosed a surgical laser scalpel with a scalpel body made of a diamond crystal, the body of which having, on the one hand, a polished scalpel blade and polished outer surfaces and, on the other hand, a laser, the laser light of which is introduced into the scalpel body via an optical fiber and predominately re-emerges from the scalpel body in the region of the scalpel blade. The emerging laser radiation in the region of the scalpel blade causes photocoagulation of the blood and thus reduces the bleeding of the wound.

In an embodiment described in FIGS. 10a and 10b, the laser axis of the optical waveguide at a coupling-in surface on a flat crystal outer surface is perpendicular to the corresponding crystal outer surface, hence the laser beam is incident on the scalpel body in a perpendicular fashion and, furthermore, the scalpel edge is situated with a lateral offset from the coupling-in surface by basically a right angle. The coupled-in laser light runs in the scalpel body up to a reflection outer surface opposite the coupling-in surface, at which reflection outer surface it is internally reflected, and it then emerges from the scalpel body at the scalpel edge. The main direction of propagation or the optical axis of the laser light is in each case changed by approximately 90° at the reflection surface and so the angle of incidence and angle of reflection of the central beam of the laser light at the reflection surface is 45°. As per FIG. 10a, the laser beam impinges on a reflection surface, which is curved in a concave fashion toward the outside and in a convex fashion toward the inside, and so the beam is widened there. If the reflection surface is selected such that it is convex toward the outside and concave toward the inside, the laser beam focus on the exit face can be focused on the scalpel edge.

In FIG. 10b, the otherwise flat crystal outer surface is designed to be concave toward the outside and convex toward the inside in the region of the coupling-in surface and it thus forms a type of lens on the surface of the scalpel body, and therefore the initially parallel laser beam is widened due to the refraction of this lens. The widened laser beam then impinges on the flat reflection surface on the other side of the scalpel-body crystal and said beam is then reflected to the laser edge from there. In FIG. 10b, reversing the design of the concave entry surface, which is convex toward the inside, into a surface that is convex toward the outside and concave toward the inside can also allow the laser beam now to be focused and then be incident on the reflection surface as a focused laser beam. On the crystal, the design of a concave or convex coupling-in surface acting like a lens is comparatively complex, just like the precise adjustment of the laser light which has to follow thereupon. The concave or convex design of the reflection surface as per FIG. 10a likewise means increased complexity when polishing the crystal.

### BRIEF SUMMARY OF THE INVENTION

Example implementations of the invention are based on the object of specifying a laser scalpel, by means of which a high proportion of the laser light coupled into the scalpel body can be provided on the scalpel edge or is able to emerge therefrom, but which scalpel is nevertheless comparatively easy to produce.

According to one implementation, the laser light (or the laser beam) may be coupled into the scalpel body at an acute angle, selected from an angular range of between 15° and 55°, with respect to the surface normal (or axis of incidence) of the coupling-in surface, that is to say with respect to the direction or axis perpendicular or normal to the coupling-in surface, unlike the prior art, where there is perpendicular incidence. The effect of this inventive idea is that there is a refraction of the laser beam toward the surface normal and so said laser beam then runs more in the direction of the scalpel body or closer to the tip thereof than before entering the scalpel body, but unlike perpendicular incidence, like in the prior art, that is to say to couple-in with an incidence axis of the laser light which is perpendicular to the coupling-in surface on the scalpel body.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 shows a laser scalpel with a scalpel body and an optical waveguide in a side view;

FIG. 2 shows the scalpel body as per FIG. 1 with dimensions in a side view;

FIG. 3 shows the scalpel body with optical fibres as per FIG. 1 in a plan view from above; and

FIG. 4 shows an additional ground tip of a scalpel body, in particular as per FIGS. 1 to 3, in a plan view from above.

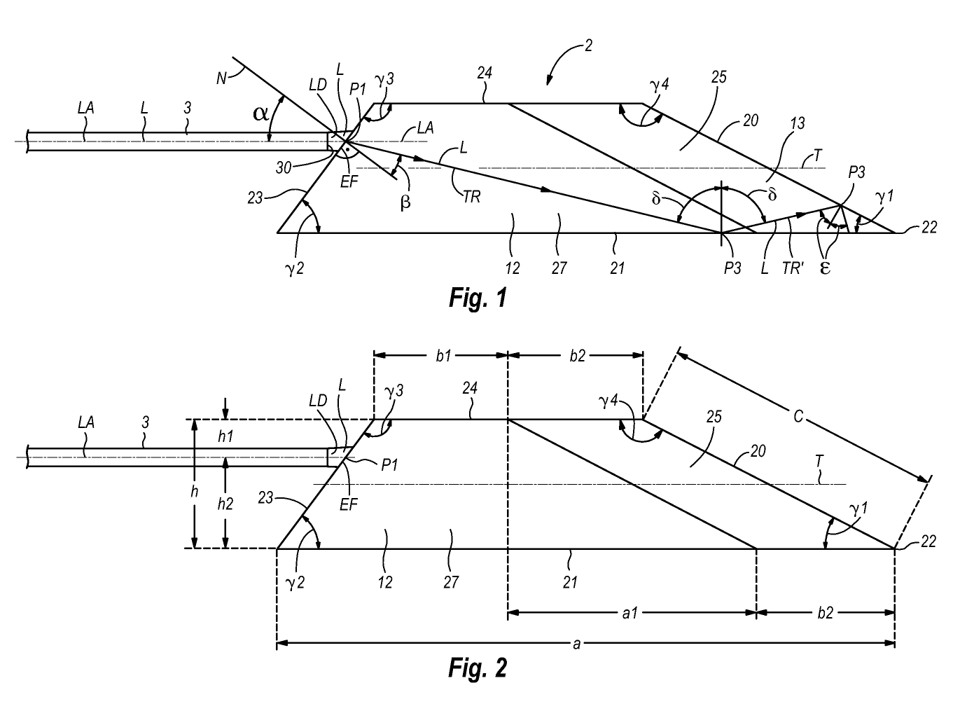

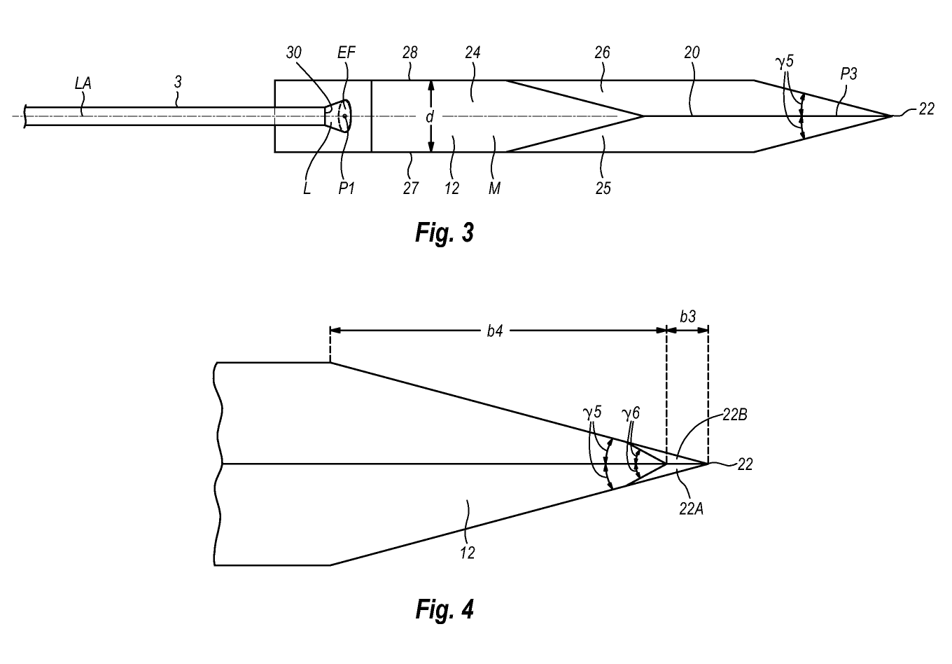

Corresponding parts and variables have been provided with the same reference signs in FIGS. 1 to 4.

### DETAILED DESCRIPTION OF THE EXAMPLE EMBODIMENTS

The laser scalpel (or laser blade, laser knife) 2 as per FIGS. 1 to 3 comprises a scalpel body 12 and an optical waveguide 3, in particular an optical fiber, for coupling laser light (or laser radiation, a laser beam) L from a laser source (not illustrated) into the scalpel body 12.

The scalpel body 12 is basically designed as an irregular polyhedron with a plurality of planar or flat polyhedral surfaces (or flat sides, outer surfaces, bevels), which together form common edges and delimit the scalpel body 12 toward the outside. A scalpel back 21, a rear side 23, a base surface 24, two side surfaces 27 and 28, and finally two cutting surface 25 and 26, which converge on a cutting edge 20, are provided as polyhedral sides of the scalpel body 12. The scalpel body 12 has a mirror-symmetrical design in respect of a central plane M. The base surface 24 and the scalpel back 21 run parallel to one another at a constant distance, which defines the height h of the scalpel body 12.

The scalpel edge formed on the front side 13 of the scalpel body 12 comprises the cutting edge 20 and the two cutting surfaces 25 and 26, which are tilted toward one another and which, on the one hand, meet at the cutting edge 20 along their longitudinal sides. Furthermore, the cutting sides 25 and 26 each form a shorter common edge with both the scalpel back 21 and the base surface 24. The scalpel edge, that is to say the cutting surfaces 25 and 26 thereof and the cutting edge 20 thereof, meets or meet (or converge with) the scalpel back 21 at a point 22 of the scalpel body 12, which point additionally is situated centrally in the central plane M. The scalpel back 21 and the cutting edge 20 subtend the acute internal angle γ1 at the tip 22, which angle is selected in particular to be between 21° and 35°, preferably to be between 25° and 30°, for example approximately 28°, and therefore the cutting edge 20 is tilted inward toward the cutting back 21.

Furthermore, the cutting surfaces 25 and 26 have, over the entire height h, the constant dimension b2, measured in the longitudinal direction. In other words, the cutting surfaces 25 and 26 are respectively shaped like a parallelogram, which is displaced in parallel by the length a1. The length c of the cutting edge 20 is given by c=a1/cos γ1=h/sin γ1 due to geometric relationships. The length b1 of the base surface 24 to the initial point of the oblique, ground cutting-surfaces 25 and 26 is designated by b1, and b2 designates the additional length of the base surface 24, which then is shaped like an equilateral triangle because of the obliquely inwardly running cutting surfaces 25 and 26, as shown in FIG. 2.

The cutting surfaces 25 and 26 of the scalpel edge and their common edges with the scalpel back 21 subtend an acute internal angle of γ5, which is e.g. 30°, with respect to the central plane M and are thus inclined inward toward the central plane M and toward one another, or are oblique. As a result, the cutting surfaces subtend the double internal angle 2·γ5, which at the same time forms the wedge angle or the internal angle of the scalpel edge.

Furthermore, the scalpel back 21 connects the tip 22 of the cutting edge to the rear side 23, which likewise is designed as a flat side or planar surface, and said back and the rear side 23 subtend the acute internal angle γ2 along their common edge, that is to say the rear side 23 is tilted obliquely toward the scalpel back 21. On its top side, the rear side 23 is connected to the scalpel edge via the base surface 24, and said rear side and the base surface 24 subtend the obtuse internal angle γ3 along their common edge. At the common corner, the base surface 24 and the cutting edge 20 in turn subtend the obtuse angle γ4. Thus, in the central plane M of the scalpel body 12, the scalpel body 12 forms the trapezium, which can be seen in FIG. 1, with the internal angles γ1, γ2, γ3 and γ4 and the side edges formed by the cutting edge 20, the scalpel back 21, the rear side 23 and the base surface 24. γ3=180°−γ2 and γ1+γ4=180° hold true because the base surface 24 and the scalpel back 21 are surfaces which run parallel to one another.

The scalpel back 21 is connected to the base surface 24 via the two side surfaces 27 and 28 in the back region and via the cutting surfaces 25 and 26 in the front region. The two side surfaces 27 and 28 run parallel to one another at the distance d, which corresponds to the width of the scalpel body 12 perpendicular to the central plane M thereof, and said surfaces are parallel to the central plane M and orthogonal to the scalpel back 21.

The scalpel body 12 is now made of a material that transmits the laser light L or is transparent thereto in at least a predetermined transmission spectrum or transmission wavelength band and, on the other hand, has the required mechanical properties in order to form the scalpel edge and be able to serve as a surgical or mechanical scalpel or blade. In particular, the material should be sufficiently hard and stable, and it should also have good sharpening or polishing properties. A crystalline material, preferably a single crystalline material, such as a sapphire or a diamond is a preferred material.

The transmission spectrum of the scalpel body 12 generally lies within a wavelength band of between 380 nm and 1200 nm, which comprises the visible spectral band and a partial band of the infrared band. The wavelength or wavelength spectrum of the laser light L lies within the transmission spectrum of the scalpel body 12 and so the latter lets the laser light L pass.

In particular, the scalpel body 12 is produced by grinding a crystal, by grinding individual flat sides or bevels into the crystal. It is understood that the shape of the cutting edge 20 is idealized as a precisely straight-lined edge. When observed under the microscope, a polished cutting edge 20 deviates from the ideal shape, which is geometrically linear, and it can have microscopically small irregularities such as undulations, etc. In this case, it suffices if the cutting edge 20 is basically smooth and continuous in comparison with the human or animal body tissue to be cut in this case, and the edge thus also can be considered to be linear.

Thus, the scalpel body 12 is shaped like a surgical scalpel, blade, or knife, which is suitable for surgery or surgical work on humans or animals, with cuts being undertaken with the scalpel edge.

The laser light L is used to coagulate or clot the blood emanating during the cutting and thus it is used to close the wound or the tissue in situ. To this end, a proportion of the laser light L, which is as large as possible and which emanates from the end 30 of the optical waveguide 3, should reach the region of the scalpel edge through the scalpel body 12; preferably, a proportion, which is as large as possible thereof, should in turn reach the vicinity of the tip 22. On the rear side 23 of the scalpel body 12, provision is made for a coupling-in surface EF for coupling the laser light L from the optical waveguide 3 and into said body.

In general, the scalpel body 12 is fastened to a hand support (not illustrated), which the surgeon holds during surgery. The optical fiber or the optical waveguide 3 is also attached to this hand holder and is optically adjusted and fixed in respect of the coupling-in surface EF on the rear side 23 of the scalpel body 12.

The optical axis of the optical waveguide 3 forms a main propagation direction of the laser light L, along which the laser light L propagates in the optical waveguide 3; said axis is referred to as the laser axis LA. The laser axis LA is parallel to the scalpel back 21 and preferably also runs in the central plane M of the scalpel body 12. When the laser light L emanates from the end 30 of the optical waveguide 3 situated at a distance from the rear side 23 of the scalpel body 12, the laser beam widens in a laser divergence region LD according to a divergence that can be quantified by the numerical aperture of the optical waveguide 3.

In this light divergence region LD, the laser light L propagates freely through the air (or a different gas) and then is incident on the surface of the oblique rear side 23 of the scalpel body 12 in the partial region that forms the coupling-in surface EF. Since the rear side 23 of the scalpel body 12 has a flat or planar design, the coupling-in surface EF is also basically situated in a plane that is at an angle to the laser axis LA. By way of example, in the case of a substantially circular cross section of the end 30 of the optical waveguide or of a conical shape of the laser divergence region LD, the coupling-in surface EF is elliptical as a result of the oblique projection onto the rear side 23.

The laser axis LA impinges on the surface of the rear side 23 at a point P1. The point P1 is situated approximately in the centre of the coupling-in surface EF. The surface normal N, which is perpendicular to the rear side 23, is indicated on the planar rear side 23 at this point P1. The propagation direction of the laser beam L, or the laser axis LA thereof, and this surface normal N subtend a coupling-in or entry angle α, which defines the oblique position between the laser axis LA and the rear side 23. In particular, the entry angle is α=90°−γ2.

Due to a change in the refractive index (or refractivity) between the surrounding air and the higher refractive index in the scalpel body 12, the laser light L is refracted by the coupling surface EF when entering or coupling into the scalpel body 12; to be precise, it is refracted toward the surface normal N due to the laws of refraction and so, after the entry into the scalpel body 12, the transmission angle β between the laser axis LA and the transmission direction TR of the laser light L within the scalpel body 12 is smaller than the entry angle α. Here, as a result of the refractive laws, the transmission angle β is an unambiguous result of the entry angle α and depends on the ratio of the usually wavelength-dependent refractive indices n1 of the air, on the one hand, and n2 of the scalpel body 12 on the other hand, according to n1·sin α=n2·sin β.

The entry angle α is generally selected between 15° and 55°, in particular in a region of between 25° and 45°, preferably in a region of between 30° and 40°, and very preferably from a region of between 34° and 36°.

It was found in most crystalline materials, particularly in the case of sapphire, that the scalpel body 12 loses comparatively little laser light L due to reflection on the coupling-in surface EF in the case of such an entry angle α between the laser axis LA and the surface normal N, that is to say the irradiated laser light L is utilized efficiently.

In the case of the geometry illustrated in the figures, a preferred value for the entry angle α was found to be 35°±1°, particularly if the scalpel body 12 is made of sapphire, because the result of this is an advantageous power distribution of the power of the laser light L on the scalpel edge and the scalpel back.

By way of example, in the case of a commercially available sapphire, this resulted in, at 810 nm and rounded to the nearest whole number, a transmission angle β=15° at the entry angle α=35° and a transmission angle β=17° at the entry angle α=40°, a transmission angle β=19° at the entry angle α=45°, a transmission angle β=13° at the entry angle α=30° and a transmission angle β=11° at the entry angle α=25°. In FIG. 1, a transmission angle β of 22° is illustrated in an exemplary fashion.

The point P1, situated in the centre of the coupling-in surface EF, is preferably also situated on the central plane M because the laser axis LA is preferably adjusted such that it comes to rest in the central plane M of the scalpel body 12. That is to say the laser light L is coupled into the scalpel body 12 at the centre.

The point of impact P1 of the laser axis LA within the coupling-in surface EF on the rear side 23 now is separated from the base surface 23 by a height h1 and separated from the scalpel back 21 by a height h2, which is greater than the height h1. h1+h2=h and h2>h1 hold true. That is to say the point P1, the entire coupling-in surface EF and the laser axis LA are situated closer to the base surface 24 than the scalpel back 21, or are above a horizontal separation plane T of the scalpel body 12, which plane runs parallel to the base surface 24 and the scalpel back 21, runs between the latter two surfaces, respectively at the same distance h/2, and is preferably directed along or perpendicular to the central plane M.

Thus, while the tip 22 of the scalpel body 12 is situated on one side (the lower side in FIG. 1) of this separation plane T, at a distance of h/2, the intersection point P1 of the laser axis LA and the rear side 23 of the scalpel body 12 is situated above the separation plane T and is separated therefrom by a distance h2−h/2; the coupling-in surface EF is also situated at least predominantly, preferably completely, above the separation plane T.

After the laser light L has entered the scalpel body 12 at the coupling-in surface EF, the laser light L propagates through the scalpel body 12 at a different transmission angle β depending on the entry point within the entry surface EF and the main propagation direction of the laser light L prevailing there in the laser divergence region LD. FIG. 1 only plots the transmission angle β and the transmission direction TR within the scalpel body 12 for the central laser beam L running along the laser axis LA and through the point P1.

Due to the unchanging refractive index, the laser light L propagates linearly within the scalpel body 12 and it meets the outer surface of the scalpel body 12 at the scalpel back 21, for example at the point P2 for the central laser beam, where the laser light L again meets the air and the lower refractive index thereof. Due to the relatively large angle of incidence δ at the point P2, the laser light L is basically completely reflected toward the inside and, to all intents and purposes, remains completely within the scalpel body 12 due to this substantially total internal reflection and it now runs along the transmission direction TR′, rotated by 180°-2δ, within the scalpel body 12 up to the next external point P3, which is now already situated in the cutting region on the cutting edge 20. There, the angle of incidence ε, which equals the angle of reflection ε, again reflects part of the laser light L internally but, depending on the magnitude of the angle ε, part of the laser light already escapes from the edge to the outside.

Thus, the reflection and transmission of the laser light L in the edge region on the front side 13, in the vicinity of the tip 22, continues and so the laser light L basically escapes outward at the tip 22 and the adjoining cutting region around the cutting edge 20 and the cutting surfaces 25 and 26 and said laser beam penetrates into the body tissue surrounding it during surgery or the surgical intervention. Therefore, the laser light L can coagulate or clot the emanating blood precisely in the region in which cuts are performed with the laser scalpel 2, namely in the region of the scalpel edge, and hence the laser light can stop the bleeding in situ directly during the intervention.

The central plane M of the scalpel body 12 is preferably placed (or the crystal, in particular sapphire, is polished) such that a growth axis or preferred crystallographic axis, such as the so-called c-axis of the crystal, is situated centrally in the central plane M or parallel thereto, is in particular a central optical axis of the crystal, which is the line of intersection between the central plane M and the separation plant T, and comes to rest parallel to the scalpel back 21 because the transmission of the crystal is particularly high in this direction.

The dimensions (rounded to one decimal place) can be selected from the following preferred value ranges:

1.2 mm≦h≦5 mm, in particular 2.5 mm≦h≦3.9 mm, preferably h=3.7 mm;

0.5 mm≦h1≦1.6 mm, in particular 0.9 mm≦h1≦1.3 mm, preferably h1=1.1 mm;

0.7 mm≦h2≦4.5 mm, in particular 1.2 mm≦h2≦3 mm, preferably h2=2.6 mm;

0.1 h2≦h1≦0.8 h2, in particular 0.3 h2≦h1≦0.6 h2, preferably h1=0.4 h2;

10 mm≦a≦25 mm, in particular 15 mm≦a≦19.5 mm, preferably a=17.3 mm;

5 mm≦a1≦10 mm, in particular 6 mm≦a1≦8.2 mm, preferably a1=7.1 mm;

1.5 mm≦b1≦7 mm, in particular 2.2 mm≦b1≦5.5 mm, preferably b1=3.7 mm;

1.5 mm≦b2≦7 mm, in particular 2.2 mm≦b2≦5.5 mm, preferably b2=3.7 mm; and

1 mm≦d≦3.5 mm, in particular 1.4 mm≦b2≦2.8 mm, preferably d=2.1 mm;

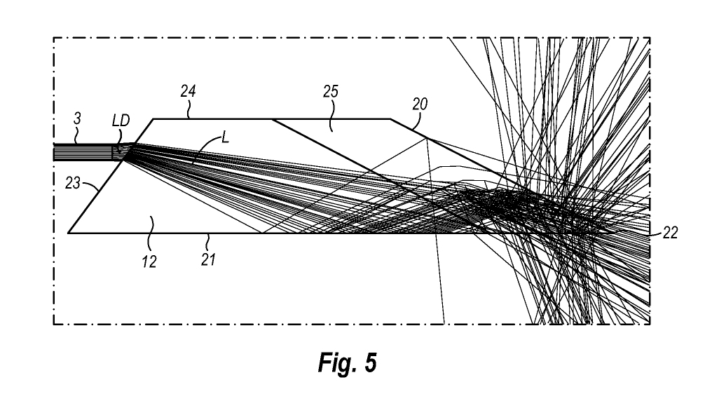

In FIG. 4, an additional polished tip 22 with laterally polished pointed surfaces 22a and 22b is generated and it has, compared to the original tip b3, a tip that is offset by b3. The length of the tip to the new tip is denoted by b4 and the internal angle of the new tip is denoted by γ6.

FIG. 5 shows a beam path of the laser light L for a scalpel body as per FIGS. 1 to 3, which beam path was calculated by a simulation. It can be seen that most of the laser light L actually reaches the cutting region on the cutting edge 20, in particular the region of the tip 22, and re-emerges from the scalpel body 12 at said location. There basically is substantially total internal reflection on the scalpel back 21 before the laser light L then reaches the cutting region or the scalpel edge on the front side 13 of the scalpel body 12. This is a result of the non-central arrangement of the optical waveguide 3 and the coupling-in surface EF with respect to the separation plane T. The angle of incidence α at the point P1 in respect of the coupling-in surface EF and its distance h2 from the scalpel back 21, and the distance a between the tip 22 and the rear side 23 or even the point P1 itself, are set such that basically the one internal reflection, in particular in or around the point P2, suffices. Here, the point P2 is a result of the transmission angle β, fixed by the refractive indices and the entry angle α, and the surface of the scalpel back 21 running parallel to the laser axis A, with the point P2 being situated close enough to the front side 13 and the scalpel edge, in particular the tip 22, that is to say the tip 22 should not be situated too far from the point P2 or the rear side 23.

It is conventional for the entry angle α to be optimized first in order to obtain a degree of transmission that is as high as possible when coupling-in the laser light L at the coupling-in surface EF and then for the geometry. In particular, the length a and the arrangement of the scalpel back 21 relative to the scalpel edge, in particular to the cutting edge 20 and point 22, to be selected and optimized such that there is only one internal reflection (at the point P2) and the vast majority of the laser light L, after running through the scalpel body 12, re-emerges from the scalpel body 12 in the region of the scalpel edge, in particular in the vicinity of the tip 22.

The present invention can be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

### LIST OF REFERENCE SIGNS

2 Scalpel
3 Optical waveguide
12 Scalpel body
13 Front side
20 Cutting edge
21 Scalpel back
22 Tip
23 Rear side
24 Base surface
25, 26 Cutting surface
27, 28 Side surface
30 End
T Separating plane
M Central plane
N Surface normal
L Laser radiation
LA Laser axis
LD Laser divergence region
EF Coupling-in surface
P1, P2, P3 Points
TR Transmission direction
α Entry angle
β Transmission angle
δ, ε Angle
γ1 First tip angle
γ2, γ3, γ4 Internal angle
γ5 Second tip angle
h, h1, h2 Height
a, a1 Length
b1, b2 Length
b3, b4 Length
d Width

We claim:

1. A laser scalpel, comprising:
a scalpel body made of a material that transmits laser radiation of at least one specific wavelength band, wherein:
the scalpel body has a scalpel edge and a scalpel back, and the scalpel edge and the scalpel back converge at a point;
the scalpel body has a coupling-in surface for coupling in laser radiation on a rear side facing away from the scalpel edge; and
the coupling-in surface is arranged and dimensioned such that part of the laser radiation coupled in on the coupling-in surface is at least in part reflected towards the scalpel edge by internal reflection on the scalpel back and a further part of the laser radiation coupled in on the coupling-in surface propagates directly through the scalpel body to the scalpel edge thereof or to the tip thereof.

2. A laser scalpel, comprising:
a scalpel body made of a material that transmits laser radiation of at least one specific wavelength band, wherein:
the scalpel body has a scalpel edge and a scalpel back, and the scalpel edge and the scalpel back converge at a point;
the scalpel body furthermore has a coupling-in surface for coupling in laser radiation on a rear side facing away from the scalpel edge; and
the coupling-in angle for the laser radiation is selected to be between 15° and 55° at the coupling-in surface.

3. The laser scalpel according to claim 2, wherein the coupling-in angle for the laser radiation is selected to be between 25° and 45° on the coupling-in surface.

4. The laser scalpel according to claim 2, wherein the coupling-in angle for the laser radiation is selected to be between 30° and 40° on the coupling-in surface.

5. The laser scalpel according to claim 2, wherein the coupling-in angle for the laser radiation is selected to be between 34° and 36° on the coupling-in surface.

6. The laser scalpel according to claim 1, wherein the rear side of the scalpel body, or at least the coupling-in surface on the rear side of the scalpel body, has a flat or planar design.

7. The laser scalpel according to claim 2, wherein the rear side of the scalpel body, or at least the coupling-in surface on the rear side of the scalpel body, has a flat or planar design.

8. A laser scalpel, comprising:
a scalpel body made of a material that transmits laser radiation of at least one specific wavelength band, wherein:
the scalpel body has a scalpel edge and a scalpel back, and the scalpel edge and the scalpel back converge at a point;
the scalpel body furthermore has a coupling-in surface for coupling in laser radiation on a rear side facing away from the scalpel edge; and
the coupling-in surface has a greater distance from the scalpel back than from a base surface of the scalpel body, which surface connects the scalpel edge at the end facing away from the tip to the rear side of the scalpel body.

9. The laser scalpel according to claim 1, further comprising means for coupling laser radiation into the scalpel body through the coupling-in surface, means for coupling laser radiation into the scalpel body comprise in particular an optical waveguide, in particular an optical fiber, the end of which is set at a distance from the coupling-in surface and/or the optical axis of which runs parallel to the scalpel back.

10. The laser scalpel according to claim 2, further comprising means for coupling laser radiation into the scalpel body through the coupling-in surface, means for coupling laser radiation into the scalpel body comprise in particular an optical waveguide, in particular an optical fiber, the end of which is set at a distance from the coupling-in surface and/or the optical axis of which runs parallel to the scalpel back.

11. The laser scalpel according to claim 8, further comprising means for coupling laser radiation into the scalpel body through the coupling-in surface, means for coupling laser radiation into the scalpel body comprise in particular an optical waveguide, in particular an optical fiber, the end of which is set at a distance from the coupling-in surface and/or the optical axis of which runs parallel to the scalpel back.
